(19) United States
(12) Patent Application Publication
(10) Pub. No.: US 2024/0177238 A1
(43) Pub. Date: May 30, 2024

(54) RESOURCE ALLOCATION METHOD AND APPARATUS AND STORAGE MEDIUM

(71) Applicant: Tsinghua University, Beijing (CN)

(72) Inventors: Qiao Yu, Beijing (CN); Xuan Ding, Beijing (CN); Hui Xiang, Beijing (CN)

(21) Appl. No.: 17/794,303

(22) PCT Filed: May 13, 2021
(86) PCT No.: PCT/CN2021/093489

§ 371 Date: Jul. 21, 2022

(30) Foreign Application Priority Data
Nov. 12, 2020 (CN) 202011260816.X

(51) Int. Cl. G06Q 40/08; G16H 50/70

(52) U.S. Cl. CPC G06Q 40/08; G16H 50/70

(57) ABSTRACT

The present disclosure relates to a resource allocation method, a resource allocation apparatus and a storage medium. The method comprises: constructing a comprehensive data system from medical insurance data and clinical path data, and determining effect parameters of different diseases accordingly; determining marginal effect parameters of a plurality of diseases according to the medical insurance data and the effect parameters of the plurality of diseases; determining resource allocation increments of the plurality of diseases in the whole disease spectrum according to the marginal effect parameters and increase amount of medical insurance resources. According to the resource allocation method of an embodiment of the present disclosure, the medical insurance data and the clinical path data can be used to analyze the effect parameters of the plurality of diseases, and the marginal effect parameters of the medical insurance data on the plurality of diseases are determined, so that resources can be allocated for the plurality of diseases based on the marginal effect parameters in a targeted manner. By increasing resources for diseases with good marginal treatment effects and less resource allocation, the efficiency of resource configuration can be improved, and the overall optimal configuration of limited medical insurance resources can be achieved.

### CROSS-REFERENCE TO RELATED APPLICATION

The present disclosure is a continuation of and claims priority under 35 U.S.C. § 120 to PCT Application. No. PCT/CN2021/093489, filed on May 13, 2021, which claims the benefit of a priority of Chinese Patent Application entitled Resource Allocation Method and Apparatus and Storage Medium, No. 202011260816.X, filed on Nov. 12, 2020. Both the above referenced priority documents are incorporated herein by reference in their entireties.

### TECHNICAL FIELD

The present disclosure relates to the technical field of computers, and in particular to a resource allocation method and apparatus and a storage medium.

### BACKGROUND

The payment scope of medical insurance benefits includes hospitalization, general outpatient, outpatient, chronic disease expense, etc. within the policy scope of the insured (i.e., the medical insurance catalog). The starting payment standard, the payment ratio and the maximum payment limit are formulated by localities within the specified scope. Payment items include: national basic medical insurance drug catalog, diagnosis and treatment items and the scope of medical service facilities. With the development of the medical insurance system, its coverage is expanding year by year, and resources such as the number of insured persons and medical insurance resources are also increasing, but at the same time, the spending pressure on medical insurance resources (for example, funds) is increasing year by year. How to scientifically and rationally configure limited funds and control the risk of misuse of fund resources have become key issues in the medical insurance field.

In the related art, the payment methods of medical insurance generally include payment by service item, payment by expense standard based on number of persons, total prepayment, etc. However, it is not involved that limited funds are allocated among different diseases. Therefore, it is difficult to achieve significant improvement in the treatment effect of a plurality of diseases in a case of limited resources. For example, a chronic disease has a long course of disease and requires large fund investment, while an acute disease has a short course of disease and a relatively small fund investment can produce a significant treatment effect. However, the acute disease may have too little investment, causing a poor treatment effect, thereby resulting in a poor overall cure rate. In the related art, it is difficult to accurately allocate resources for diseases with poor treatment effects. In addition, due to the large investment in some diseases, medical institutions are more inclined to provide more medical resources for such diseases, which may lead to situations such as overtreatment and misuse of medical resources and medical insurance resources, and may lead to shortage of medical insurance resources and medical resources for other diseases.

### SUMMARY

The present disclosure proposes a resource allocation method and apparatus, an apparatus and a storage medium.

An aspect of the present disclosure provides a resource allocation method, comprising: determining effect parameters of a plurality of diseases according to medical insurance data and clinical path data, wherein the effect parameters indicate a relationship between a treatment effect index and a case feature index in the clinical path data and the medical insurance data, the treatment effect index indicates a treatment effect of a case, and the case feature index indicates at least one category feature of the case; determining marginal effect parameters of the plurality of diseases according to the medical insurance data and the effect parameters of the plurality of diseases, wherein the marginal effect parameters indicate a change rate of the treatment effect index when the medical insurance data changes; determining resource allocation increments of the plurality of diseases according to the marginal effect parameters and increase amount of medical insurance resources.

In a possible implementation, the determining resource allocation increments of the plurality of diseases according to the marginal effect parameters and increase amount of medical insurance resources, comprises: determining the resource allocation increments of the plurality of diseases according to the marginal effect parameters of the plurality of diseases, the increase amount of the medical insurance resources, and at least one constraint condition, wherein the constraint condition includes minimum resource allocation amount for various diseases, and that a sum of the resource allocation increments of the plurality of diseases is the increase amount of the medical insurance resources.

In a possible implementation, the determining the resource allocation increments of the plurality of diseases according to the marginal effect parameters of the plurality of diseases, the increase amount of the medical insurance resources, and at least one constraint condition, comprises: determining first marginal effect parameters of the plurality of diseases according to the marginal effect parameters of plurality of diseases and the increase amount of the medical insurance resources; averaging the first marginal effect parameters of the plurality of diseases to obtain an average marginal effect parameter; determining a standard deviation of the first marginal effect parameters according to the first marginal effect parameters of the plurality of diseases and the average marginal effect parameter; under constraint of the constraint condition, minimizing the standard deviation to obtain the resource allocation increments of the plurality of diseases, wherein the resource allocation increments minimize the standard deviation of the first marginal effect parameters.

In a possible implementation, the determining effect parameters of a plurality of diseases according to medical insurance data and clinical path data, comprises: performing regression analysis processing on the medical insurance data, the treatment effect index and at least one case feature index to obtain the effect parameters.

In a possible implementation, the determining marginal effect parameters of the plurality of diseases according to the medical insurance data and the effect parameters of the plurality of diseases, comprises: performing derivation processing on the medical insurance data according to the treatment effect index and the effect parameters to obtain the marginal effect parameters.

In a possible implementation, the case feature index includes one or more of gender, age, marital status, disease type, disease severity index, surgery index, and drug dosage index.

An aspect of the present disclosure provides a resource allocation apparatus, comprising: an effect parameter module for determining effect parameters of a plurality of diseases according to medical insurance data and clinical path data, wherein the effect parameters indicate a relationship between a treatment effect index and a case feature index in the clinical path data and the medical insurance data, the treatment effect index indicates a treatment effect of a case, and the case feature index indicates at least one category feature of the case; a marginal effect module for determining marginal effect parameters of the plurality of diseases according to the medical insurance data and the effect parameters of the plurality of diseases, wherein the marginal effect parameters indicate a change rate of the treatment effect index when the medical insurance data changes; an allocation module for determining resource allocation increments of the plurality of diseases according to the marginal effect parameters and increase amount of medical insurance resources.

In a possible implementation, the allocation module is further used for determining the resource allocation increments of the plurality of diseases according to the marginal effect parameters of the plurality of diseases, the increase amount of the medical insurance resources, and at least one constraint condition, wherein the constraint condition includes minimum resource allocation amount for various diseases, and that a sum of the resource allocation increments of the plurality of diseases is the increase amount of the medical insurance resources.

In a possible implementation, the allocation module is further used for determining first marginal effect parameters of the plurality of diseases according to the marginal effect parameters of plurality of diseases and the increase amount of the medical insurance resources; averaging the first marginal effect parameters of the plurality of diseases to obtain an average marginal effect parameter; determining a standard deviation of the first marginal effect parameters according to the first marginal effect parameters of the plurality of diseases and the average marginal effect parameter; under constraint of the constraint condition, minimizing the standard deviation to obtain the resource allocation increments of the plurality of diseases, wherein the resource allocation increments minimize the standard deviation of the first marginal effect parameters.

In a possible implementation, the effect parameter module is further used for performing regression analysis processing on the medical insurance data, the treatment effect index and at least one case feature index to obtain the effect parameters.

In a possible implementation, the allocation module is further used for performing derivation processing on the medical insurance data according to the treatment effect index and the effect parameters to obtain the marginal effect parameters.

In a possible implementation, the case feature index includes one or more of gender, age, marital status, disease type, disease severity index, surgery index, and drug dosage index.

An aspect of the present disclosure provides a resource allocation apparatus, comprising: a processor; a memory for storing instructions executable by the processor; wherein the processor is configured to execute the above resource allocation method.

An aspect of the present disclosure provides a computer-readable storage medium on which computer program instructions are stored, the computer program instructions, when executed by a processor, implementing the above resource allocation method.

According to the resource allocation method of an embodiment of the present disclosure, medical insurance data and clinical path data of a plurality of diseases can be used to analyze effect parameters of a plurality of diseases, and then marginal effect parameters of the medical insurance data on the plurality of diseases are determined, so that resources can be allocated for the plurality of diseases based on the marginal effect parameters in a targeted manner. By increasing resources for diseases with good marginal treatment effects and less resource allocation, the efficiency of resource configuration can be improved and the overall optimal configuration of limited medical insurance resources can be achieved.

It should be understood that the above general description and the following detailed description are only exemplary and explanatory, but do not limit the present disclosure.

Other features and aspects of the present disclosure will become apparent from the following detailed description of exemplary embodiments with reference to the drawings.

### BRIEF DESCRIPTION OF THE DRAWINGS

The drawings here are incorporated into the specification and constitute a part of the specification. These drawings show embodiments conforming to the present disclosure, and are used to explain the technical solutions of the present disclosure together with the specification.

FIG. 1 shows a flowchart of a resource allocation method according to an embodiment of the present disclosure;

FIGS. 2A, 2B, 2C and 2D show schematic diagrams of application of the resource allocation method according to an embodiment of the present disclosure;

FIG. 3 shows a block diagram of a resource allocation apparatus according to an embodiment of the present disclosure;

FIG. 4 shows a block diagram of the resource allocation apparatus according to an embodiment of the present disclosure;

FIG. 5 shows a block diagram of the resource allocation apparatus according to an embodiment of the present disclosure.

### DETAILED DESCRIPTION

Various exemplary embodiments, features and aspects of the present disclosure will be described in detail below with reference to the drawings. The same reference numerals in the drawings denote elements that have the same or similar functions. While various aspects of the embodiments are shown in the drawings, the drawings are not necessarily drawn to scale unless otherwise indicated.

The word “exemplary” used exclusively herein means “serving as an example, embodiment, or illustration”. Any embodiment described herein as “exemplary” is not necessarily to be construed as preferred or advantageous over other embodiments.

The term “and/or” herein is only an association relationship to describe associated objects, and denotes that there can be three kinds of relationships. For example, A and/or B may denote that A exists alone, A and B exist at the same time, and B exists alone. In addition, the term “at least one” herein denotes any one of the plurality or any combination of at least two of the plurality. For example, including at least one of A, B, and C, may denote including any one or more elements selected from the set consisting of A, B and C.

In addition, in order to better illustrate the present disclosure, numerous specific details are given in the following detailed description. It should be understood by those skilled in the art that the present disclosure can be practiced without certain specific details. In some instances, methods, means, elements and circuits well known to those skilled in the art will not be described in detail, so as to highlight the gift of the present disclosure.

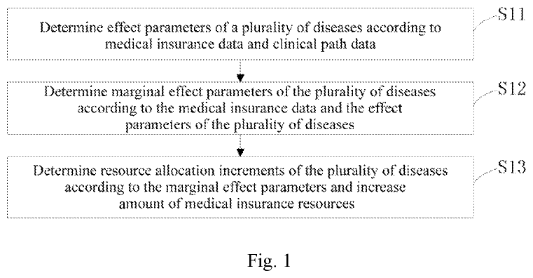

FIG. 1 shows a flowchart of a resource allocation method according to an embodiment of the present disclosure. As shown in FIG. 1, the method includes:
In step S11, effect parameters of a plurality of diseases are determined according to medical insurance data and clinical path data, wherein the effect parameters indicate a relationship between a treatment effect index and a case feature index in the clinical path data and the medical insurance data, the treatment effect index indicates a treatment effect of a case, and the case feature index indicates at least one category feature of the case;
In step S12, marginal effect parameters of the plurality of diseases are determined according to the medical insurance data and the effect parameters of the plurality of diseases, wherein the marginal effect parameters indicate a change rate of the treatment effect index when the medical insurance data changes;
In step S13, resource allocation increments of the plurality of diseases are determined according to the marginal effect parameters and increase amount of medical insurance resources.

According to the resource allocation method of an embodiment of the present disclosure, the medical insurance data and the clinical path data of the plurality of diseases can be used to analyze the effect parameters of the plurality of diseases, and then the marginal effect parameters of the medical insurance data on the plurality of diseases are determined, so that resources can be allocated for the plurality of diseases based on the marginal effect parameters in a targeted manner. By increasing resources for diseases with good marginal treatment effects and less resource allocation, the efficiency of resource configuration can be improved and the overall optimal configuration of limited medical insurance resources can be achieved.

In a possible implementation, medical insurance resources (for example, funds) are generally managed by localities, and due to reasons such as population growth, population aging, etc., the number of patients will increase, resulting in a shortage of medical insurance resources. When allocating funds for medical insurance, the allocation can be generally performed according to factors such as a historical fund requirement, the number of patients, a reimbursement ratio, etc. This allocation method does not take into account the treatment effect of the disease, which may easily lead to a case where less resources are allocated for some diseases with high cure rates. For example, if medical insurance resources are allocated according to the historical fund requirement, more medical insurance resources are allocated for chronic diseases such as diabetes and hypertension, and major diseases such as cancer, while less resources are allocated for some sudden diseases (for example, pneumonia, tuberculosis, etc.). Moreover, when the total amount of medical insurance resources increases (for example, the total amount of medical insurance resources increases due to the increase in the number of insured persons, or the total amount of medical insurance resources increases due to fiscal appropriation), the incremental part will be also inclined to allocate more funds for diseases with high historical fund requirements, resulting in more shortage of medical insurance resources and medical resources allocated for other diseases.

In addition, due to the large fund investment in chronic diseases such as diabetes and hypertension and major diseases such as cancer, medical institutions are more inclined to allocate medical resources to these diseases, which not only results in less medical resources for other diseases, but also may cause the risk of misuse of medical resources or even overtreatment in chronic diseases and major diseases. As a result, the allocation of medical resources and medical insurance resources is less uniform, and the treatment effect of diseases with limited medical resources is poor.

In a possible implementation, based on the above problems, a more accurate allocation method of medical insurance resources can be determined based on medical insurance data (for example, historical fund requirements for the treatment of various diseases, etc.) and clinical path data of the disease (for example, clinical data from multiple hospitals, which can include data of cases of a plurality of diseases, such as the treatment effect, severity, etc. of the cases). On the basis of maintaining the original allocation method of medical insurance resources, the increase amount of medical insurance resources is allocated more for diseases with a currently poor treatment effect, a less resource allocation but a better marginal effect, so as to improve the treatment effect of this disease faster, while the medical insurance resources allocated for other diseases will not reduce. In this way, the treatment effects of a plurality of diseases are more uniform, and the allocation of medical insurance resources is more equitable, thereby reducing the risks of misuse of medical resources and overtreatment.

In a possible implementation, in step S11, the effect parameters of the plurality of diseases may be determined according to the medical insurance data and the clinical path data of the plurality of diseases. In an example, a comprehensive data system may be constructed from the medical insurance data and the clinical path data. The clinical path data may include treatment data for multiple cases, such as the category of disease, medication records, surgery records, visit registration records, hospitalization registration records (for example, basic information of registrable cases, such as age, marital status, etc.), inspection reports (for example, records of medical inspection, category of lesions, severity, recovery, treatment effect of recordable cases, etc.), prescription information, etc. of multiple cases. The medical insurance data can record the hospitalization expense information, medication expense information, treatment expense information, expense settlement information, etc. of the case. The present disclosure does not limit the contents included in the medical insurance data and the clinical path data.

In a possible implementation, a complete, accurate, true and reliable comprehensive data system can be constructed from the medical insurance data and the clinical path data of a plurality of diseases. The clinical path data can be associated and integrated with the medical insurance data paid for the case. In an example, treatment expense and medical insurance payment records for each time may be recorded according to a complex data recording method, and trial balance may be performed to improve the accuracy of data. In an example, the records of clinical treatment or medication in the clinical path data and the payment records of medical insurance can be associated with each other according to methods such as byte association, and can be encrypted by a blockchain technology. The tamper-proof characteristic of blockchain technology makes the data safe and reliable, and can protect the privacy of patients. In an example, the medical insurance data can be associated with the clinical path data of multiple medical institutions in the above manner.

In a possible implementation, the effect parameters of the plurality of diseases may be determined according to the associated medical insurance data and clinical path data. In an example, the effect parameters may be determined using the treatment effect index and the case feature index of a plurality of cases, and the corresponding medical insurance data. Step S11 may include: performing regression analysis processing on the medical insurance data, the treatment effect index and at least one case feature index to obtain the effect parameters.

In an example, the case feature index includes one or more of gender, age, marital status, disease type, disease severity index, surgery index, and drug dosage index. Regression analysis can be performed using one or more of the above indexes and the payment records in the medical insurance data for the disease of the case, to obtain the effect parameter representing the relationship between the clinical path data and the medical insurance data.

In an example, regression analysis can be performed by the following formula (1) to obtain the effect parameter:

qᵢ=β₀+β₁ ln yᵢ+β₂ageᵢ+β₃genderᵢ+β₄marriageᵢ+β₅diseaseᵢ+β₆severeᵢ+β₇medicalᵢ+β₈surgeryᵢ+εᵢ (1)

Where qᵢ denotes the treatment effect index for the i-th case (qᵢ=0 denotes not cured, qᵢ=1 denotes getting better, and qᵢ=2 denotes cured), yᵢ denotes amount paid by medical insurance funds of the i-th case acquired according to the medical insurance data, ageᵢ, genderᵢ, marriageᵢ, diseaseᵢ, severeᵢ, medicalᵢ, surgeryᵢ are all case feature indexes, where ageᵢ denotes the age of the i-th case, genderᵢ denotes the gender of the i-th case, marriageᵢ denotes the marital status of the i-th case, diseaseᵢ denotes the type of disease of the i-th case (e.g., diseaseᵢ=0 denotes a common disease, diseaseᵢ=1 denotes a major disease, diseaseᵢ=2 denotes a particularly major disease such as malignant neoplasms), severeᵢ denotes severity index (e.g., severeᵢ=0 denotes general, severeᵢ=1 denotes emergency, severeᵢ=2 denotes critical), medicalᵢ denotes drug dosage index, surgeryᵢ denotes surgery index (e.g., surgeryᵢ=0 denotes no surgery is performed, surgeryᵢ=1 denotes surgery is performed), and εᵢ denotes the residual term. β₀, β₁, β₂, β₃, β₄, β₅, β₆, β₇, β₈ are the effect parameters, denoting the relationship between the treatment effect index, the case feature index and the medical insurance data.

In a possible implementation, based on the type of disease, the cases are divided according to the type of disease, and for each disease, regression analysis is performed on a plurality of cases of this disease by the above formula (1), to determine the relationship between the treatment effect index for this disease, the case feature index for this disease and the amount paid by medical insurance funds for the treatment of this disease. In an example, there can be a logarithmic relationship between the treatment effect index and the amount paid by medical insurance funds, so the amount paid by medical insurance funds can be logarithmically calculated (e.g., logarithm with e as the base) and then the regression analysis is performed by formula (1).

In this way, the relationship between the treatment effect index, the case feature index and the medical insurance data can be established through a large amount of accurate clinical path data and medical insurance data, the accuracy of the relationship is improved, and an accurate basis is provided for the allocation of the increase amount of medical insurance resources based on treatment effect.

In a possible implementation, after determining the effect parameter, that is, determining the relationship between the treatment effect index, the case feature index and the medical insurance data, the above relationship can be used to determine the marginal effect parameters of a plurality of diseases.

In a possible implementation, the effect parameter for a certain disease may denote the relationship between the treatment effect index of the disease, the case feature index of the disease and the amount paid by medical insurance funds for the treatment of the disease. This relationship may be represented by a relationship graph, and in an example, may be represented by a curve relationship graph.

In an example, the amount yⱼ paid by medical insurance funds for the j-th disease (j is a positive integer) can be used as the abscissa, the treatment effect index qⱼ can be used as the ordinate, and other parameters can be used as constants, to determine the curve relationship graph. The relationship between the amount yⱼ paid by medical insurance funds and the treatment effect index qⱼ (including the curve relationship graph and the relationship expression) can be obtained.

In a possible implementation, the marginal effect parameter of the disease (i.e. the lifting amount of the treatment effect index that can be caused by unit increase of the amount paid by medical insurance funds, or the change rate of the treatment effect index with respect to the amount paid by medical insurance funds) can be determined according to the relationship between the amount yⱼ paid by medical insurance funds and the treatment effect index qⱼ for the j-th disease. In an example, any segment of the relationship curve can be taken, and the ratio of the change amount of the treatment effect index to the change amount of the amount paid by medical insurance funds is determined to obtain the marginal effect parameter. Alternatively, multiple segments can be taken in the relationship curve, and for each segment of the curve, the ratio of the change amount of the treatment effect index to the change amount of the amount paid by medical insurance funds is determined separately. The ratios of the multiple segments of the curve are averaged to obtain the marginal effect parameter. The present disclosure does not limit the method of obtaining the marginal effect parameter.

In a possible implementation, step S12 may include: performing derivation processing on the medical insurance data according to the treatment effect index and the effect parameters to obtain the marginal effect parameters. That is, the marginal effect parameter of each disease can be obtained by performing derivation on the relationship expression between the amount yⱼ paid by medical insurance funds for each disease and the treatment effect index qⱼ for each disease. In an example, since the relationship between the treatment effect index and the logarithm of the amount paid by medical insurance funds is determined in the relationship expression, the derivation process can determine the marginal effect parameter of each disease by the following formula (2):

gⱼ′(yⱼ)=β₁ⱼ/yⱼ (2)

Where qⱼ′(yⱼ) is the marginal effect parameter of the j-th disease, β₁ⱼ is the coefficient of the amount yⱼ paid by medical insurance funds in the relationship expression of the j-th disease, that is, the change rate of the treatment effect of the j-th disease with respect to the amount paid by medical insurance funds of the j-th disease. The marginal effect parameter of each disease can be determined according to formula (2).

In a possible implementation, in step S13, after the marginal effect parameter is determined, the resource allocation amount of a plurality of diseases may be determined. In an example, due to a large number of people involved, it is difficult to change the existing allocation scheme of medical insurance resources. Therefore, the newly added medical insurance resources can be allocated by the resource allocation method, that is, the increase amount of medical insurance resources can be allocated, and in a long-term iteration, the overall allocation of medical insurance resources is optimized.

In a possible implementation, the increase amount of medical insurance resources can be optimally allocated according to the marginal effect parameters of various diseases, so as to determine the resource allocation increment of each disease, that is, share in the increase amount of medical insurance resources for each disease obtained by the allocation. Further, in a long-term iteration, the increase amount of medical insurance resources over the years can be optimally allocated, so as to achieve an overall optimal allocation of medical insurance resources. In an example, the annual increase amount of medical insurance resources can be optimally allocated through an iterative time of 7-10 years, so as to achieve the overall optimal allocation of medical insurance resources, so that the treatment effects of various diseases are more uniform, the difference between marginal effects of various diseases is smaller, the types of diseases with inefficient resource configuration are reduced, the allocation of medical insurance resources is more equitable, and the risks of misuse of medical resources and overtreatment are reduced. The present disclosure does not limit the iteration time.

In a possible implementation, step S13 may include: determining the resource allocation increments of the plurality of diseases according to the marginal effect parameters of the plurality of diseases, the increase amount of the medical insurance resources, and at least one constraint condition, wherein the constraint condition includes minimum resource allocation amount for various diseases, and that a sum of the resource allocation increments of the plurality of diseases is the increase amount of the medical insurance resources. In an example, under constraint of the constraint condition, the resource allocation increments of various diseases can be optimized according to the marginal effect parameters of various diseases. For example, the resource allocation increments of various diseases can be optimized by linear optimization, and the present disclosure does not limit the optimization method.

In an example, the constraint condition may include that the sum of the resource allocation increments of the plurality of diseases is the increase amount of the medical insurance resources, and the constraint condition may be determined by the following formula (3):

Z=Σⱼ₌₁ⁿZⱼ (3)

Where Zⱼ is the share in the increase amount of medical insurance resources allocated for the j-th disease, n is the number of disease types, and Z is the increase amount of medical insurance resources.

In an example, the constraint condition may include minimum resource allocation amount for various diseases, and may be determined by the following formula (4):

Zⱼ≥kⱼZ (4)

Where kⱼ is the smallest proportion of the resource allocation increment of the j-th disease, in an example, kⱼ can be a decimal such as 0.01, and Σⱼ₌₁ⁿkⱼ≤1.

In a possible implementation, determining the resource allocation increments of the plurality of diseases according to the marginal effect parameters of the plurality of diseases, the increase amount of the medical insurance resources, and at least one constraint condition, includes: determining first marginal effect parameters of the plurality of diseases according to the marginal effect parameters of plurality of diseases and the increase amount of the medical insurance resources; averaging the first marginal effect parameters of the plurality of diseases to obtain an average marginal effect parameter; determining a standard deviation of the first marginal effect parameters according to the first marginal effect parameters of the plurality of diseases and the average marginal effect parameter; under constraint of the constraint condition, minimizing the standard deviation to obtain the resource allocation increments of the plurality of diseases, wherein the resource allocation increments minimize the standard deviation of the first marginal effect parameters.

In a possible implementation, formula (2) can represent the marginal effect parameter of the j-th disease. On this basis, if the resource allocation increment allocated for the j-th disease is Zⱼ, the per capita allocation increment of the j-th disease is zⱼ, where Zⱼ=mⱼzⱼ, mⱼ is the number of cases of the j-th disease. Therefore, after newly adding medical insurance resources, the first marginal effect parameter of the j-th disease can be expressed by the following formula (5):

qⱼ′(yⱼ+zⱼ)=β₁ⱼ/(yⱼ+zⱼ) (5)

In a possible implementation, the resource allocation increment Zⱼ of each disease can be optimized by optimizing the first marginal effect parameter of the plurality of diseases. In an example, the standard deviation of the first marginal effect parameters can be optimized in order to reduce the difference between the marginal effect parameters of the medical insurance resources for treating various diseases.

In a possible implementation, the standard deviation of the first marginal effect parameters of a plurality of diseases can be determined. The first marginal effect parameters of a plurality of diseases can be averaged to obtain the average marginal effect parameter, which can be determined by the following formula (6) in an example:

$${Avg\_q}^{′} = \frac{1}{n} {\sum }_{j = 1}^{n} {q}_{j}^{′} ( {y}_{j} + {z}_{j} ) \qquad ( 6 )$$

Where Avg_q′ is the average marginal effect parameter.

In a possible implementation, the standard deviation of the first marginal effect parameters may be determined according to the first marginal effect parameters of a plurality of diseases and the average marginal effect parameter. In an example, the standard deviation of the first marginal effect parameters may be determined by the following formula (7):

Std=\sqrt{Σⱼ₌₁ⁿ[qⱼ′(yⱼ+zⱼ)−Avg_q′]²} (7)

Where Std is the standard deviation of the first marginal effect parameters. Under the constraint condition determined by formulas (3) and (4), the standard deviation Std of the first marginal effect parameters can be minimized, so that the difference between the marginal effect parameters of the increase amount of the medical insurance resources for treating various diseases is minimized. When the standard deviation Std of the first marginal effect parameters is minimized, the share Zⱼ* of the increase amount of medical insurance resources allocated for various diseases is the resource allocation increments of various diseases. In this case, the resource allocation increments of various diseases can minimize the standard deviation Std of the first marginal effect parameters, and therefore, the difference between the marginal effect parameters of the increase amount of the medical insurance resources for treating various diseases is minimized. That is, the treatment effects of various diseases in the whole disease spectrum are uniformed to reduce the types of diseases with inefficient resource configuration, the allocation of medical insurance resources is more equitable, and the risks of misuse of medical resources and overtreatment are reduced.

In a possible implementation, the resource allocation increment can also be used to determine expected marginal effect parameters of various diseases after allocating the increase amount of medical insurance resources. For example, the resource allocation increment Zⱼ* and formula (5) can be used to determine the expected marginal effect parameters of various diseases, that is, qⱼ′ (yⱼ+Zⱼ*/mⱼ)=β₁ⱼ/(yⱼ+Zⱼ*/mⱼ). Further, the resource allocation increment Zⱼ* and the relationship expression between the amount yⱼ paid by medical insurance funds and the treatment effect index qⱼ can also be used to determine the expected effect indexes of various diseases, that is, yⱼ+Zⱼ*/mⱼ is substituted into the position of yⱼ in the relationship expression, and the calculated effect index is the expected effect index.

According to the resource allocation method of the embodiment of the present disclosure, the medical insurance data and the clinical path data of the plurality of diseases can be used to establish the relationship between the treatment effect index, the case feature index and the medical insurance data, so as to analyze the effect parameters of the plurality of diseases, and then the marginal effect parameters of the medical insurance data on the plurality of diseases are determined, so that resources can be allocated for the plurality of diseases based on the marginal effect parameters in a targeted manner. By increasing resources for diseases with good marginal treatment effects and less resource allocation, the efficiency of resource configuration can be improved and the overall optimal configuration of limited medical insurance resources can be achieved.

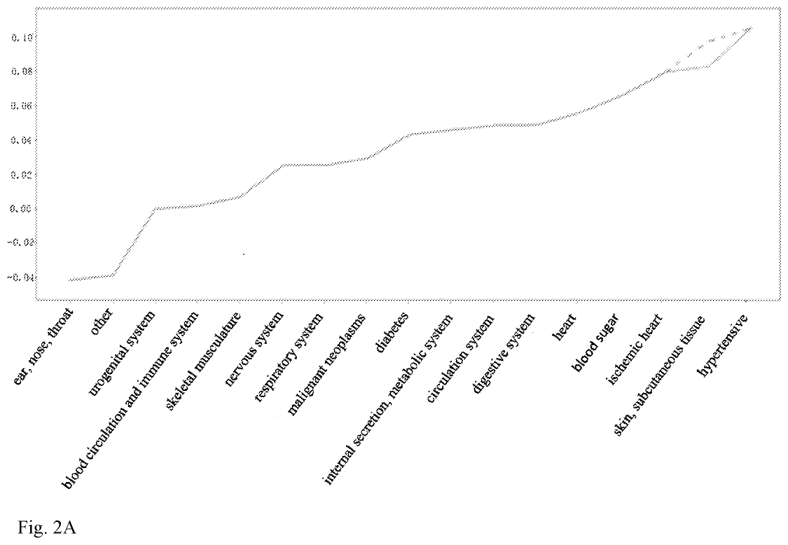

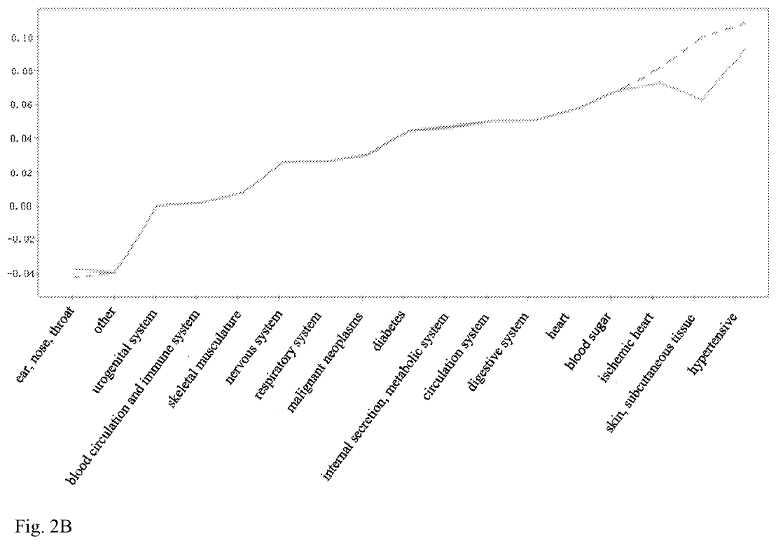

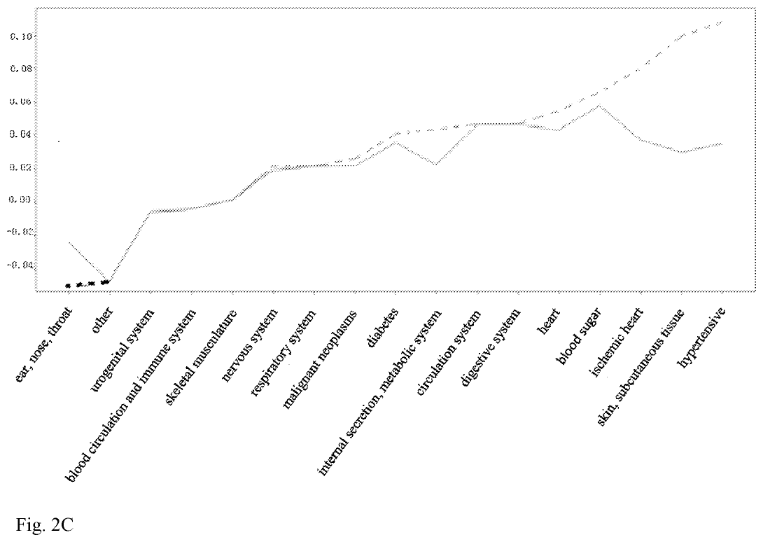

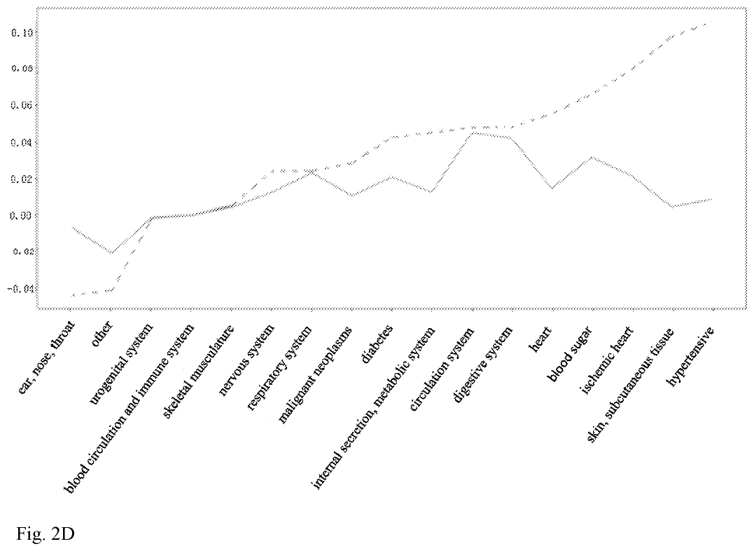

FIGS. 2A, 2B, 2C, and 2D show schematic diagrams of application of the resource allocation method according to the embodiment of the present disclosure. In an example, according to the clinical path data, the medical insurance data and formula (1), the relationship expression between the treatment effect index, the case feature index of each disease and the amount paid by medical insurance funds for the treatment of the disease can be determined.

In a possible implementation, after the above relationship expression is determined, the marginal effect parameter of each disease can be determined by formula (2). Further, when medical insurance resources increase (for example, the medical insurance funds increase), the first marginal effect parameters of various diseases can be determined by formula (5), and based on the first marginal effect parameters, the allocation share of the medical insurance resources for treating various diseases is optimized. For example, the standard deviation of the first marginal effect parameters can be obtained by formula (7), and under two constraint conditions of “the sum of resource allocation increments of a plurality of diseases is the increase amount of medical insurance resources” and “the minimum resource allocation amount for various diseases”, the standard deviation of the first marginal effect parameters is minimized. The share Zⱼ* of the increase amount of medical insurance resources allocated for various diseases, which is obtained when the standard deviation is minimized, is the resource allocation increments of various diseases.

In a possible implementation, the resource allocation increment Zⱼ* and formula (5) can be used to determine the expected marginal effect parameters of various diseases. In addition, the resource allocation increment Zⱼ*, and the relationship expression between the treatment effect index, the case feature index of each disease and the amount paid by the medical insurance funds for the treatment of the disease are used to determine the expected effect indexes of various diseases.

In an example, FIG. 2A shows the marginal effect parameters of various diseases when the increase amount of medical insurance resources is 10,000 yuan, the solid line in the figure is the marginal effect parameter of each disease when the resource allocation method is used, and the dotted line is the marginal effect parameter of each disease when the resource allocation method is not used (when the allocation is performed according to factors such as a historical fund requirement, the number of patients, a reimbursement ratio, etc.). As shown in FIG. 2A, after using the resource allocation method, the difference between the marginal effect parameters of diseases such as skin and subcutaneous tissue diseases and hypertensive disease and those of other diseases is smaller, that is, the marginal effect parameters of various diseases are more uniform. In an example, when there is no increase amount of medical insurance resources, the standard deviation of the marginal effect parameters of a plurality of diseases is 0.04226, and when the increase amount of medical insurance resources is 10,000 yuan, after using the resource allocation method, the standard deviation of the marginal effect parameters of the plurality of diseases is 0.04098. That is, the standard deviation of the marginal effect parameters is reduced and the marginal effect parameters of the plurality of diseases are more uniform.

In an example, FIG. 2B shows the marginal effect parameters of various diseases when the increase amount of medical insurance resources is 100,000 yuan, the solid line in the figure is the marginal effect parameter of each disease when the resource allocation method is used, and the dotted line is the marginal effect parameter of each disease when the resource allocation method is not used. As shown in FIG. 2B, after using the resource allocation method, the difference between the marginal effect parameters of diseases such as ear, nose and throat diseases, blood sugar disease, ischemic heart disease, skin and subcutaneous tissue diseases and hypertensive disease and those of other diseases is smaller, that is, the marginal effect parameters of various diseases are more uniform. In an example, when the increase amount of medical insurance resources is 100,000 yuan, after using the resource allocation method, the standard deviation of the marginal effect parameters of the plurality of diseases is 0.03680. That is, the standard deviation of the marginal effect parameters is reduced and the marginal effect parameters of the plurality of diseases are more uniform.

In an example, FIG. 2C shows the marginal effect parameters of various diseases when the increase amount of medical insurance resources is 1,000,000 yuan, the solid line in the figure is the marginal effect parameter of each disease when the resource allocation method is used, and the dotted line is the marginal effect parameter of each disease when the resource allocation method is not used. As shown in FIG. 2C, after using the resource allocation method, the difference between the marginal effect parameters of more types of diseases and those of other diseases is smaller, that is, the marginal effect parameters of various diseases are more uniform. In an example, when the increase amount of medical insurance resources is 1,000,000 yuan, after using the resource allocation method, the standard deviation of the marginal effect parameters of the plurality of diseases is 0.02625. That is, the standard deviation of the marginal effect parameters is reduced and the marginal effect parameters of the plurality of diseases are more uniform.

In an example, FIG. 2D shows the marginal effect parameters of various diseases when the increase amount of medical insurance resources is 10,000,000 yuan, the solid line in the figure is the marginal effect parameter of each disease when the resource allocation method is used, and the dotted line is the marginal effect parameter of each disease when the resource allocation method is not used. As shown in FIG. 2D, after using the resource allocation method, the difference between the marginal effect parameters of more types of diseases and those of other diseases is significantly reduced, that is, the marginal effect parameters of various diseases are more uniform. In an example, when the increase amount of medical insurance resources is 10,000,000 yuan, after using the resource allocation method, the standard deviation of the marginal effect parameters of the plurality of diseases is 0.02625. That is, the standard deviation of the marginal effect parameters is reduced and the marginal effect parameters of the plurality of diseases are more uniform.

As shown in FIGS. 2A-2D, with the increase in the increase amount of medical insurance resources, after using the resource allocation method, by increasing resources for diseases with good marginal treatment effects and less resource allocation, the marginal effect parameters of a plurality of diseases are more uniform, the treatment effects of a plurality of diseases are more average, the problem that the allocation of medical resources and medical insurance resources is non-uniform and the treatment effects of diseases with short medical resources are poor is alleviated, the Pareto improvement is performed on the increase amount of medical insurance resources, and the configuration efficiency of the increase amount of medical insurance resources of a plurality of diseases in the whole disease spectrum is improved, so that the allocation of medical insurance resources is more equitable, and the risks of misuse of medical resources and overtreatment are reduced.

It can be understood that the above method embodiments mentioned in the present disclosure can be combined with each other to form combined embodiments without violating the principle and logic. Due to space limitations, the present disclosure will not repeat the description thereof.

In addition, the present disclosure also provides a resource allocation apparatus, an apparatus, a computer-readable storage medium and program, all of which can be used to implement any resource allocation method provided by the present disclosure. The corresponding technical solutions and description can be known by referring to the corresponding records in the method section, and will not be repeated.

Those skilled in the art can understand that in the above method of the detailed description, the writing order of each step does not mean a strict execution order or constitute any limitation on the implementation process, and the specific execution order of each step should be determined based on its function and possible internal logic.

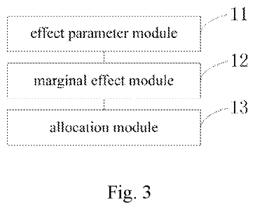

FIG. 3 shows a block diagram of a resource allocation apparatus according to an embodiment of the present disclosure. As shown in FIG. 3, the apparatus includes: an effect parameter module 11 for determining effect parameters of a plurality of diseases according to medical insurance data and clinical path data, wherein the effect parameters indicate a relationship between a treatment effect index and a case feature index in the clinical path data and the medical insurance data, the treatment effect index indicates a treatment effect of a case, and the case feature index indicates at least one category feature of the case; a marginal effect module 12 for determining marginal effect parameters of the plurality of diseases according to the medical insurance data and the effect parameters of the plurality of diseases, wherein the marginal effect parameters indicate a change rate of the treatment effect index when the medical insurance data changes; an allocation module 13 for determining resource allocation increments of the plurality of diseases according to the marginal effect parameters and increase amount of medical insurance resources.

In a possible implementation, the allocation module is further used for determining the resource allocation increments of the plurality of diseases according to the marginal effect parameters of the plurality of diseases, the increase amount of the medical insurance resources, and at least one constraint condition, wherein the constraint condition includes minimum resource allocation amount for various diseases, and that a sum of the resource allocation increments of the plurality of diseases is the increase amount of the medical insurance resources.

In a possible implementation, the allocation module is further used for determining first marginal effect parameters of the plurality of diseases according to the marginal effect parameters of plurality of diseases and the increase amount of the medical insurance resources; averaging the first marginal effect parameters of the plurality of diseases to obtain an average marginal effect parameter; determining a standard deviation of the first marginal effect parameters according to the first marginal effect parameters of the plurality of diseases and the average marginal effect parameter; under constraint of the constraint condition, minimizing the standard deviation to obtain the resource allocation increments of the plurality of diseases, wherein the resource allocation increments minimize the standard deviation of the first marginal effect parameters.

In a possible implementation, the effect parameter module is further used for performing regression analysis processing on the medical insurance data, the treatment effect index and at least one case feature index to obtain the effect parameters.

In a possible implementation, the allocation module is further used for performing derivation processing on the medical insurance data according to the treatment effect index and the effect parameters to obtain the marginal effect parameters.

In a possible implementation, the case feature index includes one or more of gender, age, marital status, disease type, disease severity index, surgery index, and drug dosage index.

In some embodiments, the functions or modules included in the apparatus provided in the embodiment of the present disclosure can be used to execute the methods described in the above method embodiments. The specific implementation can be known by referring to the description of the above method embodiments, and will not be repeated for brevity.

The embodiment of the present disclosure further provides a computer-readable storage medium on which computer program instructions are stored, the computer program instructions, when executed by a processor, implementing the above method. The computer-readable storage medium may be a non-volatile computer-readable storage medium.

The embodiment of the present disclosure further provides an apparatus, including: a processor; a memory for storing instructions executable by the processor; wherein the processor is configured to execute the above method.

The apparatus may be provided as a terminal, a server or a device of other forms.

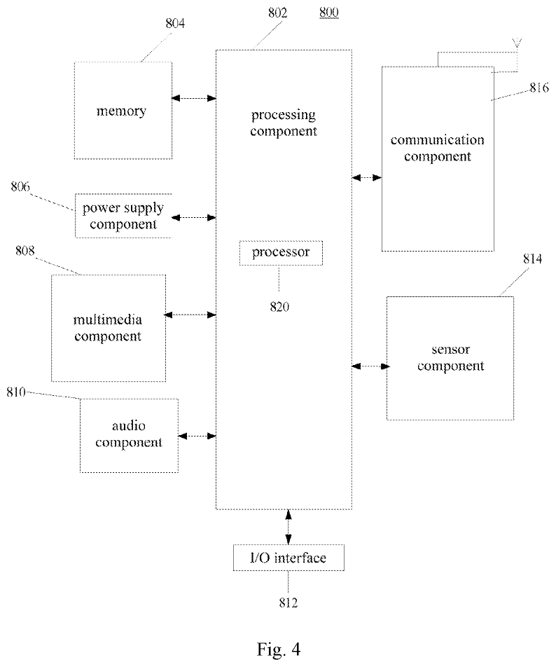

FIG. 4 is a block diagram of a resource allocation apparatus according to an exemplary embodiment. For example, an apparatus 800 may be a terminal, such as mobile phone, computer, digital broadcast terminal, messaging device, game console, tablet device, medical device, fitness device, personal digital assistant, etc.

Referring to FIG. 4, the apparatus 800 may include one or more of the following components: a processing component 802, a memory 804, a power supply component 806, a multimedia component 808, an audio component 810, an input/output (I/O) interface 812, a sensor component 814, and a communication component 816.

The processing component 802 generally controls the overall operation of the apparatus 800, such as operations associated with display, phone call, data communication, camera operation, and recording operation. The processing component 802 may include one or more processors 820 to execute instructions to complete all or some of the steps of the above method. In addition, the processing component 802 may include one or more modules to facilitate interaction between the processing component 802 and other components. For example, the processing component 802 may include a multimedia module to facilitate interaction between the multimedia component 808 and the processing component 802.

The memory 804 is configured to store various types of data to support operations at the apparatus 800. Examples of such data include instructions for any application program or method operating on the apparatus 800, contact data, phonebook data, messages, pictures, videos, etc. The memory 804 may be implemented by any type of volatile or non-volatile storage device or a combination thereof, such as static random access memory (SRAM), electrically erasable programmable read only memory (EEPROM), erasable programmable read only memory (EPROM), programmable read only memory (PROM), read only memory (ROM), magnetic memory, flash memory, magnetic disk or optical disk.

The power supply component 806 provides power to the various components of the apparatus 800. The power components 806 may include a power supply management system, one or more power supplies, and other components associated with generation, management, and allocation of power for the apparatus 800.

The multimedia component 808 includes a screen that provides an output interface between the apparatus 800 and a user. In some embodiments, the screen may include a liquid crystal display (LCD) and a touch panel (TP). If the screen includes a touch panel, the screen may be implemented as a touch screen to receive input signals from the user. The touch panel includes one or more touch sensors to sense touch, slide, and gestures on the touch panel. The touch sensor may not only sense the boundary of a touch or slide action, but also detect duration and pressure associated with the touch or slide action. In some embodiments, the multimedia component 808 includes a front camera and/or a rear camera. When the apparatus 800 is in an operation mode, such as a shooting mode or a video mode, the front camera and/or the rear camera may receive external multimedia data. Each of the front camera and the rear camera may be a fixed optical lens system or have a focal length and an optical zoom capability.

The audio component 810 is configured to output and/or input audio signals. For example, the audio component 810 includes a microphone (MIC) that is configured to receive external audio signals when the apparatus 800 is in an operation mode, such as a call mode, a recording mode, and a voice recognition mode. The received audio signals may be further stored in the memory 804 or transmitted via the communication component 816. In some embodiments, the audio component 810 also includes a speaker for outputting audio signals.

The I/O interface 812 provides an interface between the processing component 802 and a peripheral interface module, which may be a keyboard, a click wheel, a button, or the like. These buttons may include, but are not limited to: a home button, a volume button, a start button, and a lock button.

The sensor component 814 includes one or more sensors for providing the apparatus 800 with state evaluation of various aspects. For example, the sensor component 814 can detect an on/off state of the apparatus 800 and the relative positioning of components, such as a display and a keypad of the apparatus 800. The sensor component 814 can also detect position change of the apparatus 800 or a component of the apparatus 800, the presence or absence of user contact with the apparatus 800, the orientation or acceleration/deceleration of the apparatus 800 and temperature change of the apparatus 800. The sensor component 814 may include a proximity sensor configured to detect the presence of objects nearby without any physical contact. The sensor component 814 may also include a light sensor, such as a CMOS or CCD image sensor, for use in imaging application. In some embodiments, the sensor component 814 may also include an acceleration sensor, a gyroscope sensor, a magnetic sensor, a pressure sensor, or a temperature sensor.

The communication component 816 is configured to facilitate wired or wireless communication between the apparatus 800 and other devices. The apparatus 800 may access wireless networks based on communication standards, such as WiFi, 2G or 3G, or a combination thereof. In an exemplary embodiment, the communication component 816 receives broadcast signals or broadcast related information from an external broadcast management system via a broadcast channel. In an exemplary embodiment, the communication component 816 also includes a near field communication (NFC) module to facilitate short-range communication. For example, the NFC module can be implemented based on radio frequency identification (RFID) technology, infrared data association (IrDA) technology, ultra-wideband (UWB) technology, Bluetooth (BT) technology and other technologies.

In an exemplary embodiment, the apparatus 800 may be implemented by one or more application specific integrated circuits (ASICs), digital signal processors (DSPs), digital signal processing devices (DSPDs), programmable logic devices (PLDs), field-programmable gate arrays (FPGAs), controllers, microcontrollers, microprocessors or other electronic elements for executing the above method.

In an exemplary embodiment, a non-volatile computer-readable storage medium, such as the memory 804 including computer program instructions, is also provided. The computer program instructions are executable by the processor 820 of the apparatus 800 to complete the above method.

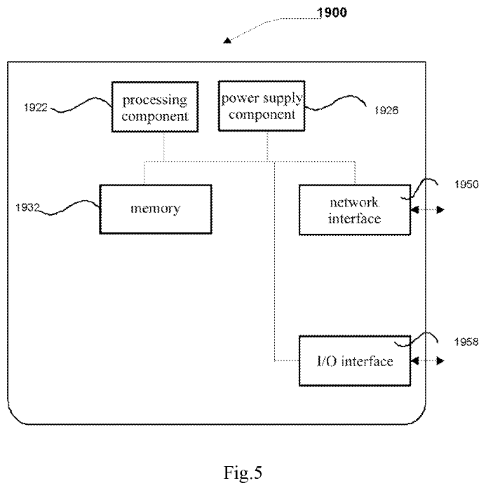

FIG. 5 is a block diagram of a resource allocation apparatus according to an exemplary embodiment. For example, an apparatus 1900 may be provided as a server. Referring to FIG. 5, the apparatus 1900 includes a processing component 1922 which further includes one or more processors, and a memory resource represented by a memory 1932 for storing instructions executable by the processing component 1922, such as application program. The application program stored in memory 1932 may include one or more modules, each corresponding to a set of instructions. In addition, the processing component 1922 is configured to execute instructions to execute the above method.

The apparatus 1900 may also include a power supply component 1926 configured to execute power supply management of the apparatus 1900, a wired or wireless network interface 1950 configured to connect the apparatus 1900 to a network, and an input output (I/O) interface 1958. The apparatus 1900 may operate based on an operating system stored in the memory 1932, such as Windows Server™, Mac OS X™, Unix™, Linux™, FreeBSD™ or the like.

In an exemplary embodiment, a non-volatile computer-readable storage medium, such as the memory 1932 including computer program instructions, is also provided. The computer program instructions are executable by the processing component 1922 of the apparatus 1900 to complete the above method.

The present disclosure may be implemented by a system, a method, and/or a computer program product. The computer program product may include a computer readable storage medium having computer readable program instructions for causing a processor to carry out the aspects of the present disclosure stored thereon.

The computer readable storage medium can be a tangible device that can retain and store instructions used by an instruction executing device. The computer readable storage medium may be, but not limited to, e.g., electronic storage device, magnetic storage device, optical storage device, electromagnetic storage device, semiconductor storage device, or any proper combination thereof. A non-exhaustive list of more specific examples of the computer readable storage medium includes: portable computer diskette, hard disk, random access memory (RAM), read-only memory (ROM), erasable programmable read-only memory (EPROM or Flash memory), static random access memory (SRAM), portable compact disc read-only memory (CD-ROM), digital versatile disc (DVD), memory stick, floppy disk, mechanically encoded device (for example, punch-cards or raised structures in a groove having instructions recorded thereon), and any proper combination thereof. A computer readable storage medium referred herein should not to be construed as transitory signal per se, such as radio waves or other freely propagating electromagnetic waves, electromagnetic waves propagating through a waveguide or other transmission media (e.g., light pulses passing through a fiber-optic cable), or electrical signal transmitted through a wire.

Computer readable program instructions described herein can be downloaded to individual computing/processing devices from a computer readable storage medium or to an external computer or external storage device via network, for example, the Internet, local area network, wide area network and/or wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium in the respective computing/processing devices.

Computer readable program instructions for carrying out the operations of the present disclosure may be assembler instructions, instruction-set-architecture (ISA) instructions, machine instructions, machine-related instructions, microcode, firmware instructions, state-setting data, or source code or object code written in any combination of one or more programming languages, including an object oriented programming language, such as Smalltalk, C++ or the like, and the conventional procedural programming languages, such as the “C” programming language or similar programming languages. The computer readable program instructions may be executed completely on the user's computer, partly on the user's computer, as a stand-alone software package, partly on the user's computer and partly on a remote computer, or completely on a remote computer or a server. In the scenario with remote computer, the remote computer may be connected to the user's computer through any type of network, including local area network (LAN) or wide area network (WAN), or connected to an external computer (for example, through the Internet connection from an Internet Service Provider). In some embodiments, electronic circuitry, such as programmable logic circuitry, field-programmable gate arrays (FPGA), or programmable logic arrays (PLA), may be customized from state information of the computer readable program instructions; the electronic circuitry may execute the computer readable program instructions, so as to achieve the aspects of the present disclosure.

Aspects of the present disclosure have been described herein with reference to the flowchart and/or the block diagrams of the method, device (systems), and computer program product according to the embodiments of the present disclosure. It will be appreciated that each block in the flowchart and/or the block diagram, and combinations of blocks in the flowchart and/or block diagram, can be implemented by the computer readable program instructions.

These computer readable program instructions may be provided to a processor of a general purpose computer, a dedicated computer, or other programmable data processing devices, to produce a machine, such that the instructions create means for implementing the functions/acts specified in one or more blocks in the flowchart and/or block diagram when executed by the processor of the computer or other programmable data processing devices. These computer readable program instructions may also be stored in a computer readable storage medium, wherein the instructions cause a computer, a programmable data processing device and/or other devices to function in a particular manner, such that the computer readable storage medium having instructions stored therein comprises a product that includes instructions implementing aspects of the functions/acts specified in one or more blocks in the flowchart and/or block diagram.

The computer readable program instructions may also be loaded onto a computer, other programmable data processing devices, or other devices to have a series of operational steps performed on the computer, other programmable devices or other devices, so as to produce a computer implemented process, such that the instructions executed on the computer, other programmable devices or other devices implement the functions/acts specified in one or more blocks in the flowchart and/or block diagram.

The flowcharts and block diagrams in the drawings illustrate the architecture, function, and operation that may be implemented by the system, method and computer program product according to the various embodiments of the present disclosure. In this regard, each block in the flowchart or block diagram may represent a part of a module, a program segment, or a portion of code, which comprises one or more executable instructions for implementing the specified logical function(s). In some alternative implementations, the functions denoted in the blocks may occur in an order different from that denoted in the drawings. For example, two contiguous blocks may, in fact, be executed substantially concurrently, or sometimes they may be executed in a reverse order, depending upon the functions involved. It will also be noted that each block in the block diagram and/or flowchart, and combinations of blocks in the block diagram and/or flowchart, can be implemented by dedicated hardware-based systems performing the specified functions or acts, or by combinations of dedicated hardware and computer instructions

Although the embodiments of the present disclosure have been described above, it will be appreciated that the above descriptions are merely exemplary, but not exhaustive; and that the disclosed embodiments are not limiting. A number of variations and modifications may occur to one skilled in the art without departing from the scopes and spirits of the described embodiments. The terms in the present disclosure are selected to provide the best explanation on the principles and practical applications of the embodiments and the technical improvements to the arts on market, or to make the embodiments described herein understandable to one skilled in the art.

What is claimed is:

1. A resource allocation method, comprising:
by at least one processor, determining effect parameters of a plurality of diseases based on medical insurance data and clinical path data, wherein the effect parameters indicate a relationship of a treatment effect index with a case feature index in the clinical path data and the medical insurance data, the treatment effect index indicates a treatment effect of a case, and the case feature index indicates at least one category feature of the case;
by the at least one processor, determining marginal effect parameters of the plurality of diseases based on the medical insurance data and the effect parameters of the plurality of diseases, wherein the marginal effect parameters indicate a change rate of the treatment effect index when the medical insurance data changes;
by the at least one processor, determining resource allocation increments of the plurality of diseases based on the marginal effect parameters and an increase amount of medical insurance resources.

2. The method according to claim 1, wherein the determining resource allocation increments of the plurality of diseases based on the marginal effect parameters and the increase amount of medical insurance resources, comprises:
determining the resource allocation increments of the plurality of diseases based on the marginal effect parameters of the plurality of diseases, the increase amount of the medical insurance resources, and at least one constraint condition, wherein the constraint condition includes a minimum resource allocation amount for various diseases, and that a sum of the resource allocation increments of the plurality of diseases is the increase amount of the medical insurance resources.

3. The method according to claim 2, wherein the determining the resource allocation increments of the plurality of diseases based on the marginal effect parameters of the plurality of diseases, the increase amount of the medical insurance resources, and at least one constraint condition, comprises:
determining first marginal effect parameters of the plurality of diseases based on the marginal effect parameters of plurality of diseases and the increase amount of the medical insurance resources;
averaging the first marginal effect parameters of the plurality of diseases to obtain an average marginal effect parameter;
determining a standard deviation of the first marginal effect parameters based on the first marginal effect parameters of the plurality of diseases and the average marginal effect parameter;
under constraint of the constraint condition, minimizing the standard deviation to obtain the resource allocation increments of the plurality of diseases, wherein the resource allocation increments minimize the standard deviation of the first marginal effect parameters.

4. The method according to claim 1, wherein the determining effect parameters of a plurality of diseases based on medical insurance data and clinical path data, comprises:
performing regression analysis processing on the medical insurance data, the treatment effect index, and at least one case feature index to obtain the effect parameters.

5. The method according to claim 4, wherein the determining marginal effect parameters of the plurality of diseases based on the medical insurance data and the effect parameters of the plurality of diseases, comprises:
performing derivation processing on the medical insurance data based on the treatment effect index and the effect parameters to obtain the marginal effect parameters.

6. The method according to claim 1, wherein the case feature index includes one or more of gender, age, marital status, disease type, disease severity index, surgery index, and drug dosage index.

7. A resource allocation apparatus, comprising:
at least one processor;
at least one memory for storing instructions executable by the at least one processor;
wherein the at least one processor is configured to execute the method of:
determining effect parameters of a plurality of diseases based on medical insurance data and clinical path data, wherein the effect parameters indicate a relationship of a treatment effect index with a case feature index in the clinical path data and the medical insurance data, the treatment effect index indicates a treatment effect of a case, and the case feature index indicates at least one category feature of the case;
determining marginal effect parameters of the plurality of diseases based on the medical insurance data and the effect parameters of the plurality of diseases, wherein the marginal effect parameters indicate a change rate of the treatment effect index when the medical insurance data changes;
determining resource allocation increments of the plurality of diseases based on the marginal effect parameters and an increase amount of medical insurance resources.

8. The method according to claim 7, wherein the determining resource allocation increments of the plurality of diseases based on the marginal effect parameters and the increase amount of medical insurance resources, comprises:
determining the resource allocation increments of the plurality of diseases based on the marginal effect parameters of the plurality of diseases, the increase amount of the medical insurance resources, and at least one constraint condition, wherein the constraint condition includes a minimum resource allocation amount for various diseases, and that a sum of the resource allocation increments of the plurality of diseases is the increase amount of the medical insurance resources.

9. The method according to claim 8, wherein the determining the resource allocation increments of the plurality of diseases based on the marginal effect parameters of the plurality of diseases, the increase amount of the medical insurance resources, and at least one constraint condition, comprises:
determining first marginal effect parameters of the plurality of diseases based on the marginal effect parameters of plurality of diseases and the increase amount of the medical insurance resources;
averaging the first marginal effect parameters of the plurality of diseases to obtain an average marginal effect parameter;
determining a standard deviation of the first marginal effect parameters based on the first marginal effect parameters of the plurality of diseases and the average marginal effect parameter;
under constraint of the constraint condition, minimizing the standard deviation to obtain the resource allocation increments of the plurality of diseases, wherein the resource allocation increments minimize the standard deviation of the first marginal effect parameters.

10. The method according to claim 7, wherein the determining effect parameters of a plurality of diseases based on medical insurance data and clinical path data, comprises:
performing regression analysis processing on the medical insurance data, the treatment effect index, and at least one case feature index to obtain the effect parameters.

11. The method according to claim 10, wherein the determining marginal effect parameters of the plurality of diseases based on the medical insurance data and the effect parameters of the plurality of diseases, comprises:
performing derivation processing on the medical insurance data based on the treatment effect index and the effect parameters to obtain the marginal effect parameters.

12. The method according to claim 7, wherein the case feature index includes one or more of gender, age, marital status, disease type, disease severity index, surgery index, and drug dosage index.

13. A non-transitory computer-readable storage medium on which computer program instructions are stored, wherein the computer program instructions, when executed by at least one processor, implement the method of:
determining, by at least one processor, effect parameters of a plurality of diseases based on medical insurance data and clinical path data, wherein the effect parameters indicate a relationship of a treatment effect index with a case feature index in the clinical path data and the medical insurance data, the treatment effect index indicates a treatment effect of a case, and the case feature index indicates at least one category feature of the case;
determining, by the at least one processor, marginal effect parameters of the plurality of diseases based on the medical insurance data and the effect parameters of the plurality of diseases, wherein the marginal effect parameters indicate a change rate of the treatment effect index when the medical insurance data changes;
determining, by the at least one processor, resource allocation increments of the plurality of diseases based on the marginal effect parameters and an increase amount of medical insurance resources.

14. The method according to claim 13, wherein the determining resource allocation increments of the plurality of diseases based on the marginal effect parameters and the increase amount of medical insurance resources, comprises:
determining the resource allocation increments of the plurality of diseases based on the marginal effect parameters of the plurality of diseases, the increase amount of the medical insurance resources, and at least one constraint condition, wherein the constraint condition includes a minimum resource allocation amount for various diseases, and that a sum of the resource allocation increments of the plurality of diseases is the increase amount of the medical insurance resources.

15. The method according to claim 14, wherein the determining the resource allocation increments of the plurality of diseases based on the marginal effect parameters of the plurality of diseases, the increase amount of the medical insurance resources, and at least one constraint condition, comprises:
determining first marginal effect parameters of the plurality of diseases based on the marginal effect parameters of plurality of diseases and the increase amount of the medical insurance resources;
averaging the first marginal effect parameters of the plurality of diseases to obtain an average marginal effect parameter;
determining a standard deviation of the first marginal effect parameters based on the first marginal effect parameters of the plurality of diseases and the average marginal effect parameter;
under constraint of the constraint condition, minimizing the standard deviation to obtain the resource allocation increments of the plurality of diseases, wherein the resource allocation increments minimize the standard deviation of the first marginal effect parameters.

16. The method according to claim 13, wherein the determining effect parameters of a plurality of diseases based on medical insurance data and clinical path data, comprises:
performing regression analysis processing on the medical insurance data, the treatment effect index, and at least one case feature index to obtain the effect parameters.

17. The method according to claim 16, wherein the determining marginal effect parameters of the plurality of diseases based on the medical insurance data and the effect parameters of the plurality of diseases, comprises:
performing derivation processing on the medical insurance data based on the treatment effect index and the effect parameters to obtain the marginal effect parameters.

18. The method according to claim 13, wherein the case feature index includes one or more of gender, age, marital status, disease type, disease severity index, surgery index, and drug dosage index.
